(19) United States
(12) Patent Application Publication
(10) Pub. No.: US 2023/0257299 A1
(43) Pub. Date: Aug. 17, 2023

(54) GLASS CERAMICS AND GLASS COMPOSITE COMPOSITIONS

(71) Applicant: Ming Chi University of Technology, new Taipei City (TW)

(72) Inventors: Yu-Jie Wu, New Taipei City (TW); Guan-Yi Hung, Taipei City (TW); Pin-Yi Chen, New Taipei City (TW); Kuei-Chih Feng, Taoyuan City (TW); Chi-Shun Tu, Kaohsiung City (TW); Chi-Yun Wang, Taoyuan City (TW)

(73) Assignee: Ming Chi University of Technology, New Taipei City (TW)

(21) Appl. No.: 18/109,404

(22) Filed: Feb. 14, 2023

(30) Foreign Application Priority Data
Feb. 17, 2022 (TW) 111105825

(51) Int. Cl. C03C 10/00; C03B 19/06

(52) U.S. Cl. CPC C03C 10/0009; C03B 19/06; C03C 2204/00

(57) ABSTRACT

A glass ceramic manufactured by sequentially performing the processes of melting and thermal decomposition, water quenching and sintering of a glass composite. The glass ceramic includes 38 wt % to 49 wt % CaO, 41 wt % to 52 wt % SiO₂ and 0.1 wt % to 20 wt % P₂O₅. The glass composite includes a glass component and P₂O₅, and the glass component includes CaCO₃ and SiO₂ and does not include an alkali metal oxide. The melting and thermal composition temperature is from 1350° C. to 1650° C. The sintering temperature is from 750° C. to 1050° C. By the combination of CaO, SiO₂ and P₂O₅ and the control of the contents of CaO, SiO₂ and P₂O₅ within the aforementioned ranges, and the glass ceramic contains no alkali metal oxide, the glass ceramic has good mechanical strength and low cytotoxicity.

### TECHNICAL FIELD OF THE DISCLOSURE

The present disclosure relates to glass ceramics, and more particularly to a glass ceramic that does not contain any alkali metal and a glass composite that forms the glass ceramic.

### DESCRIPTION OF RELATED ART

Bioglass®45S5 is a biocompatible glass ceramic composed of 22.5 wt % to 26.5 wt % CaO, 43 wt % to 47 wt % SiO₂, 22.5 wt % to 26.5 wt % Na₂O and 5 wt % to 7 wt % P₂O₅. The Bioglass®45S5 can be used as a filling material for bone defect or a repair material for periodontal tissue defect, etc. However, the Bioglass®45S5 has poor mechanical properties and is not conducive to be used as a fixing bracket for defect tissue repairs. In addition, the Bioglass®45S5 has a high sodium content which causes cytotoxicity issues.

### SUMMARY OF THE DISCLOSURE

Therefore, it is a first objective of this disclosure to provide a glass ceramic with good mechanical property and low cytotoxicity.

In this disclosure, the glass ceramic is manufactured by a method, and the method includes the steps of sequentially performing a melting and thermal decomposition, a water quenching process, a forming process and a sintering process of a glass composite, and when the total quantity of glass ceramic is considered as 100 wt %, the glass ceramic includes 38 wt % to 49 wt % CaO, 41 wt % to 52 wt % SiO₂ and 0.1 wt % to 20 wt % P₂O₅, and the glass composite includes a glass component and P₂O₅. The glass component includes CaCO₃ and SiO₂ and does not include an alkali metal oxide. The melting and thermal decomposition temperature falls within a range from 1350° C. to 1650° C. The sintering temperature falls within a range from 750° C. to 1050° C.

A second objective of this disclosure is to provide a glass composite.

In this disclosure, the glass composite includes a glass component and P₂O₅. The glass component includes CaCO₃ and SiO₂ and does not include an alkali metal oxide. When the total quantity of glass component is considered as 100 wt %, the content of CaCO₃ falls within a range from 55 wt % to 65 wt %. When the total quantity of glass component is considered as 100 parts by weight, the content of P₂O₅ falls within a range from 0.1 part by weight to 18 parts by weight.

This disclosure has the following effects: By the combination of CaO, SiO₂ and P₂O₅, the control of the content of CaO in a range from 38 wt % to 49 wt %, the control of the content of SiO₂ in a range from 41 wt % to 52 wt %, and the control of the content of P₂O₅ in a range from 0.1 wt % to 20 wt %, the glass ceramic has good compressive strength, and good flexural strength. Since the glass composite does not contain any alkali metal oxide, therefore the glass ceramic formed by the glass composite has low cytotoxicity and good biocompatibility.

### BRIEF DESCRIPTION OF THE DRAWINGS

The technical characteristics of this disclosure will become apparent with the detailed description of preferred embodiments accompanied with the illustration of related drawings.

FIGS. 1 and 2 are scanning electron microscopy (SEM) images showing the surface densities of the glass ceramics in accordance with the Embodiments 58 to 61 and Embodiments 63 to 69 of this disclosure; and

FIG. 3 is an X-ray diffraction pattern showing the crystal structures of the glass ceramics in accordance with Embodiments 58 to 69 of this disclosure.

### DETAILED DESCRIPTION OF THE DISCLOSURE

This disclosure will be descripted in details below

Glass Ceramic

In this disclosure, the glass ceramic is manufactured by a method, which includes the steps of sequentially performing a melting and thermal decomposition, a water quenching process and a sintering process of a glass composite, and the glass ceramic includes 38 wt % to 49 wt % CaO, 41 wt % to 52 wt % SiO₂ and 0.1 wt % to 20 wt % P₂O₅ when the total quantity of glass ceramic is considered as 100 wt %; wherein the glass composite includes a glass component and P₂O₅, and the glass component comprises CaCO₃ and SiO₂ and do not contains any alkali metal oxide, and the melting and thermal decomposition temperature falls within a range from 1350° C. to 1650° C., and the sintering temperature falls within a range from 750° C. to 1050° C.

The melting and thermal decomposition is intended for setting the CaCO₃, SiO₂ and P₂O₅ in the glass composite to a melted state, so that they can be mixed uniformly, as well as changing the CaCO₃ into CaO to obtain a melt mixture containing CaO, SiO₂ and P₂O₅. In some implementation modes of this disclosure, the melting and thermal decomposition time falls within a range from 2 hours to 10 hours.

The water quenching process is intended for changing the melt mixture into a plurality of amorphous quenched glass lumps with a glassy phase. In detail, the cooling speed of the melt mixture is greater than the crystallization speed in the water quenching process, so that the melt mixture will be solidified to form amorphous quenched glass lumps before entering into the crystalline phase.

The sintering process is intended for changing the amorphous quenched glass lumps with a glassy phase and the crystalline glass ceramic. In some implementation modes of this disclosure, the sintering time falls within a range from 0.5 hour to 4 hours. In some implementation modes of this disclosure, the glass ceramic has at least one crystal structure, and the crystal structure is one selected from the group consisting of an alpha-wollastonite (α-CaSiO₃) crystal structure, a beta-wollastonite (β-CaSiO₃) crystal structure or a hydroxyapatite [Ca₅(PO₄)₃(OH)] crystal structure.

In some implementation modes of this disclosure, the method further includes the step of performing a grinding process of the amorphous quenched glass lumps in the presence of at least one liquid medium to form a plurality of glass cullets with a substantially uniform size. The liquid medium includes but not limited to water or ethanol, etc. In some implementation modes of this disclosure, the liquid medium is one selected from the group consisting of water and ethanol. In some implementation modes of this disclosure, each glass cullet has a size smaller than 50 μm.

In some implementation modes of this disclosure, in order to fit the size or shape of different defect parts or meet the requirement of mechanical strength for different defect parts when the glass ceramic is used as a biomedical filling material in the biomedical field, the method further includes the step of performing a forming process of the glass cullets in the presence of an adhesive, so that the glass cullets stick together to form a plurality of glass lumps. The glass lumps include but not limited to glass ingots with a size greater than 50 μm and smaller than 1 mm. In some implementation modes of this disclosure, the adhesive is one selected from the group consisting of polyvinyl alcohol, polyvinyl butyral, carboxyethyl cellulose, and sodium carboxymethyl cellulose.

In some implementation modes of this disclosure, when the sintering temperature is 750° C. and the total quantity of glass ceramic is considered as 100 wt %, the content of P₂O₅ falls within a range from 3.9 wt % to 14.2 wt % in order to make the glass ceramic have an appropriate compressive strength, and when the total quantity of glass ceramic is considered as 100 wt %, the content of P₂O₅ falls within a range from 5.2 wt % to 10 wt % in order to make the glass ceramic have a better compressive strength.

In some implementation modes of this disclosure, when the sintering temperature is 750° C. and the total quantity of glass ceramic is considered as 100 wt %, the content of P₂O₅ falls within a range from 3.9 wt % to 17.2 wt %, in order to make the glass ceramic be conducive to osteoblast differentiation, and when the total quantity of glass ceramic is considered as 100 wt %, the content of P₂O₅ falls within a range from 7.6 wt % to 13.2 wt % in order to make the glass ceramic be more conducive to osteoblast differentiation.

In some implementation modes of this disclosure, when the sintering temperature is 950° C. and the total quantity of glass ceramic is considered as 100 wt %, the content of P₂O₅ falls within a range from 3.9 wt % to 18.1 wt % in order to make the glass ceramic have an appropriate compressive strength, and when the total quantity of glass ceramic is considered as 100 wt %, the content of P₂O₅ falls within a range from 8.8 wt % to 14.2 wt % in order to make the glass ceramic have a better compressive strength.

In some implementation modes of this disclosure, when the sintering temperature is 950° C. and the total quantity of glass ceramic is considered as 100 wt %, the content of P₂O₅ falls within a range from 2.6 wt % to 15.2 wt % in order to make the glass ceramic be conducive to osteoblast differentiation. When the total quantity of glass ceramic is considered as 100 wt %, the content of P₂O₅ falls within a range from 5.2 wt % to 12.2 wt % in order to make the glass ceramic be more conducive to osteoblast differentiation.

In some implementation modes of this disclosure, when the sintering temperature is 1050° C. and the total quantity of glass ceramic is considered as 100 wt %, the content of P₂O₅ falls within a range from 3.9 wt % to 14.2 wt % in order to make the glass ceramic have an appropriate compressive strength; and when the total quantity of glass ceramic is considered as 100 wt %, the content of P₂O₅ falls within a range from 5.2 wt % to 13.2 wt %, in order to make the glass ceramic have a better compressive strength.

In some implementation modes of this disclosure, when the sintering temperature is 1050° C. and the total quantity of glass ceramic is considered as 100 wt %, the content of P₂O₅ falls within a range from 0.1 wt % to 16.2 wt % in order to make the glass ceramic be conducive to osteoblast differentiation; and when the total quantity of glass ceramic is considered as 100 wt %, the content of P₂O₅ falls within a range from 5.2 wt % to 13.2 wt % in order to make the glass ceramic be more conducive to osteoblast differentiation.

Glass Composite

When the total quantity of glass component is considered as 100 wt %, the content of CaCO₃ falls within a range from 55 wt % to 65 wt %. When the total quantity of glass component is considered as 100 parts by weight, the content of P₂O₅ falls within a range from 0.1 part by weight to 18 parts by weight.

In some implementation modes of this disclosure, the total quantity of glass component is considered as 100 wt %, the content of CaCO₃ is 62.49 wt %.

In some implementation modes of this disclosure, when the sintering temperature is 750° C. and the total quantity of glass component is considered as 100 parts by weight, the content of P₂O₅ falls within a range from 3 parts by weight to 12 parts by weight in order to make the glass ceramic have an appropriate compressive strength; and when the total quantity of glass component is considered as 100 parts by weight, the content of P₂O₅ falls within a range from 4 parts by weight to 8 parts by weight in order to make the glass ceramic have a better compressive strength.

In some implementation modes of this disclosure, when the sintering temperature is 750° C., and the total quantity of glass component is considered as 100 parts by weight, the content of P₂O₅ falls within a range from 3 parts by weight to 15 parts by weight in order to make the glass ceramic be conducive to osteoblast differentiation; and when the total quantity of glass component is considered as 100 parts by weight, the content of P₂O₅ falls within a range from 6 parts by weight to 11 parts by weight to make the glass ceramic be more conducive to osteoblast differentiation.

In some implementation modes of this disclosure, when the sintering temperature is 950° C. and the total quantity of glass component considered as 100 parts by weight, the content of P₂O₅ falls within a range from 3 parts by weight to 16 parts by weight in order to make the glass ceramic have an appropriate compressive strength; and when the total quantity of glass component is considered as 100 parts by weight, the content of P₂O₅ falls within a range from 7 parts by weight to 12 parts by weight in order to make the glass ceramic have a better compressive strength.

In some implementation modes of this disclosure, when the sintering temperature is 950° C. and the total quantity of glass component is considered as 100 parts by weight, the content of P₂O₅ falls within a range from 2 parts by weight to 13 parts by weight in order to make the glass ceramic be conducive to osteoblast differentiation. When the total quantity of glass component is considered as 100 parts by weight, the content of P₂O₅ falls within a range from 4 parts by weight to 10 parts by weight in order to make the glass ceramic be more conducive to osteoblast differentiation.

In some implementation modes of this disclosure, when the sintering temperature is 1050° C. and the total quantity of glass component is considered as 100 parts by weight, the content of P₂O₅ falls within a range from 3 parts by weight to 12 parts by weight in order to make the glass ceramic have an appropriate compressive strength. When the total quantity of glass component is considered as 100 parts by weight, the content of P₂O₅ falls within a range from 4 parts by weight to 11 parts by weight in order to make the glass ceramic have a better compressive strength.

In some implementation modes of this disclosure, when the sintering temperature is 1050° C. and the total quantity of glass component is considered as 100 parts by weight, the content of P₂O₅ falls within a range from 0.1 part by weight to 14 parts by weight in order to make the glass ceramic be conducive to osteoblast differentiation. When the total quantity of glass component is considered as 100 parts by weight, the content of P₂O₅ falls within a range from 4 parts by weight to 11 parts by weight in order to make the glass ceramic be more conducive to osteoblast differentiation.

In some implementation modes of this disclosure, when the total quantity of glass component is considered as 100 parts by weight, the content of P₂O₅ falls within a range from 0.5 part by weight to 10 parts by weight in order to make the glass ceramic have a dense surface. In some implementation modes of this disclosure, when the total quantity of glass component is considered as 100 parts by weight, the content of P₂O₅ falls within a range from 3.5 parts by weight to 5.5 parts by weight in order to make the glass ceramic have a denser surface.

The glass ceramic of this disclosure can be used as a biomedical filling material in the biomedical field. The biomedical filling material includes but not limited to a bone filling material or an alveolar bone filling material, etc.

Exemplary embodiments are illustrated in referenced figures of the drawings. It is intended that the embodiments and figures disclosed herein are to be considered illustrative rather than restrictive.

### [Embodiment 1] Glass Composite and Glass Ceramic

62.49 wt % CaCO₃ and 37.51 wt % SiO₂ are mixed to obtain a glass component, and then 100 parts by weight of the glass component and 0.1 part by weight of P₂O₅ are mixed and then a ball milling process is performed to obtain a plurality of glass powder, and the glass powder is put into an oven to perform at a drying process 150° C. to obtain a glass composite.

The glass composite is put into a furnace with a temperature set at 1500° C. to perform a melting and thermal decomposition for 3 hours to obtain a clarified melted mixture containing CaO, SiO₂ and P₂O₅, and then the clarified melted mixture is quickly removed from the furnace and poured into deionized water at 25° C. to perform a water quenching process in order to obtain a plurality of amorphous quenched glass lumps, and then the amorphous quenched glass lumps is mixed with water and ethanol, and a zirconium ball is used for a grinding process to obtain a plurality of glass cullets with a particle size smaller than 50 μm, and then the glass cullets are mixed with polyvinyl alcohol to perform a forming process in order to obtain a plurality of glass ingots with a size greater than 50 μm and smaller than 1 mm, and then the glass ingots are sintered at 750° C. for 2 hours to obtain a glass ceramic containing 48.21 wt % CaO, 51.65 wt % SiO₂ and 0.14 wt % P₂O₅.

### Embodiments 2 to 19

The methods used in Embodiments 2 to 19 are substantially the same as the method of Embodiment 1, except that the consumption of P₂O₅ is changed.

### [Embodiment 20] Glass Composite and Glass Ceramic

62.49 wt % CaCO₃ and 37.51 wt % SiO₂ are mixed to obtain a glass component, and then 100 parts by weight of the glass component and 0.1 part by weight of P₂O₅ are mixed, and a ball milling process is performed to obtain a plurality of glass powder, and then the glass powder is put into an oven, and a drying process is performed at 150° C. to obtain a glass composite.

The glass composite is put into a furnace set at 1500° C., and a melting and thermal decomposition is performed for 3 hours to obtain a clarified melted mixture containing CaO, SiO₂ and P₂O₅, and the clarified melted mixture is quickly removed from the furnace and poured into deionized water at 25° C., and a water quenching process is performed to obtain a plurality of amorphous quenched glass lumps, and then the amorphous quenched glass lumps are mixed with water and ethanol, and a zirconium ball is used for a grinding process to obtain a plurality of glass cullets with a particle size smaller than 50 μm, and the glass cullets are mixed with polyvinyl alcohol, and a forming process is performed to obtain with a plurality of glass ingots with a size greater than 50 μm and smaller than 1 mm, and then the glass ingots are sintered at 950° C. for 2 hours to obtain a glass ceramic containing 48.21 wt %, CaO, 51.65 wt % SiO₂ and 0.14 wt % P₂O₅.

### Embodiments 21 to 38

The methods used in Embodiments 21 to 38 are substantially the same as the method of Embodiment 20, except that the consumption of P₂O₅ is changed.

### [Embodiment 39] Glass Composite and Glass Ceramic

62.49 wt % CaCO₃ and 37.51 wt % SiO₂ are mixed to obtain a glass component, and then 100 parts by weight of the glass component and 0.1 part by weight of P₂O₅ are mixed, and a ball milling process is performed to obtain a plurality of glass powder, and then the glass powder is put into an oven, and a drying process is performed at 150° C. to obtain a glass composite.

The glass composite is put into a furnace set to 1500° C., and a melting and thermal decomposition is performed for 3 hours to obtain a clarified melted mixture containing CaO, SiO₂ and P₂O₅, and then the clarified melted mixture is quickly removed from the furnace and poured into deionized water at 25° C., and a water quenching process is performed to obtain a plurality of amorphous quenched glass lumps, and then the amorphous quenched glass lumps are mixed with water and ethanol, and a zirconium ball is used to perform a crushing process to obtain a plurality of glass cullets with a particle size smaller than 50p, and then the glass cullets are mixed with polyvinyl alcohol, and a forming process is performed to obtain a plurality of glass ingots with a size greater than 50 μm and smaller than 1 mm, and then the glass ingots are sintered at 1050° C. for 2 hours to obtain a glass ceramic containing 48.21 wt % CaO, 51.65 wt % SiO₂ and 0.14 wt % P₂O₅.

### Embodiment 40 to 57

The methods used in Embodiments 40 to 57 are substantially the same as the method of Embodiment 39, except that the consumption of P₂O₅ is changed.

### [Embodiment 58] Glass Composite and Glass Ceramic

62.49 wt % CaCO₃ and 37.51 wt % SiO₂ are mixed to obtain a glass component, and then 100 parts by weight of the glass component and 0.5 part by weight of P₂O₅ are mixed, and a ball milling process is performed to obtain a plurality of glass powder, and then the glass powder is put into an oven, and a drying process is performed at 150° C. to obtain a glass composite.

The glass composite is put into a furnace set to 1500° C., and a melting and thermal decomposition is performed for 3 hours to obtain a clarified melted mixture containing CaO, SiO₂ and P₂O₅, and then the clarified melted mixture is quickly removed from the furnace and poured into deionized water at 25° C., and a water quenching process is performed to obtain a plurality of amorphous quenched glass lumps, and then the amorphous quenched glass lumps are mixed with water and ethanol, and a zirconium ball is used to perform a crushing process to obtain a plurality of glass cullets with a particle size smaller than 50 μm, and then the glass cullets are mixed with polyvinyl alcohol, and a forming process is performed to obtain a plurality of glass ingots with a size greater than 50 μm and smaller than 1 mm, and then the glass ingots are sintered at 1000° C. for 2 hours to obtain a glass ceramic containing 47.95 wt % CaO, 51.37 wt % SiO₂ and 0.68 wt % P₂O₅.

### Embodiments 59 to 69

The methods used in Embodiments 59 to 69 are substantially the same as the method of Embodiment 58, except that the consumption of P₂O₅ is changed, and the consumptions of P₂O₅ in Embodiments 59 to 69 are 1 parts by weight, 2.5 parts by weight, 3 parts by weight, 3.5 parts by weight, 4 parts by weight, 4.5 parts by weight, 5 parts by weight, 5.5 parts by weight, 6 parts by weight, 7.5 parts by weight and 10 parts by weight respectively.

### [Comparative Example 1] Glass Composite and Glass Ceramic

33.48 wt % CaCO₃, 34.46 wt % SiO₂ and 32.07 wt % Na₂CO₃ are mixed to obtain a glass component, and then 100 parts by weight of the glass component and 4.59 parts by weight of P₂O₅ are mixed, and a ball milling process is performed to obtain a plurality of glass powder, and then the glass powder is put into an oven, and a drying process is performed at 150° C. to obtain a glass composite.

The glass composite is put into a furnace which is set to 1500° C., and a melting and thermal decomposition is performed for 3 hours to obtain a clarified melted mixture containing CaO, SiO₂, Na₂O and P₂O₅, and then the clarified melted mixture is quickly removed from the furnace and poured into deionized water at 25° C., and a water quenching process is performed to obtain a plurality of amorphous quenched glass lumps, and then the amorphous quenched glass lumps are mixed with water and ethanol, and a zirconium ball is used to perform a crushing process to obtain a plurality of glass cullets with a particle size smaller than 50 μm, and then the glass cullets are mixed with polyvinyl alcohol, and a forming process is performed to obtain a plurality of glass ingots with a size greater than 50 μm and smaller than 1 mm, and the glass ingots are sintered at 750° C. for 2 hours to obtain a glass ceramic containing 24.5 wt % CaO, 45 wt % SiO₂, 24.5 wt % Na₂O and 6 wt % P₂O₅.

### Comparative Example 2 to 3

The methods used in Comparative Example 2 to 3 are substantially the same as the method of Comparative Example 1, except that the consumption of CaCO₃, SiO₂ and Na₂CO₃ in the glass component is changed.

### [Comparative Example 4] Glass Composite and Glass Ceramic

33.48 wt % CaCO₃, 34.46 wt % SiO₂ and 32.07 wt % Na₂CO₃ are mixed to obtain a glass component, and then 100 parts by weight of the glass component and 4.59 parts by weight of P₂O₅ are mixed, and a ball milling process is performed to obtain a plurality of glass powder, and then the glass powder is put into an oven, and a drying process is performed at 150° C. to obtain a glass composite.

The glass composite is put into a furnace which is set to 1500° C., and a melting and thermal decomposition is performed for 3 hours to obtain a clarified melted mixture containing CaO, SiO₂, Na₂O and P₂O₅, and then the clarified melted mixture is quickly removed from the furnace and poured into deionized water at 25° C., and a water quenching process is performed to obtain a plurality of amorphous quenched glass lumps, and then the amorphous quenched glass lumps are mixed with water and ethanol, and a zirconium ball is used to perform a crushing process to obtain a plurality of glass cullets with a particle size smaller than 50 μm, and then the glass cullets are mixed with polyvinyl alcohol, and a forming process is performed to obtain a plurality of glass ingots with a size greater than 50 μm and smaller than 1 mm, and the glass ingots are sintered at 950° C. for 2 hours to obtain a glass ceramic containing 24.5 wt % CaO, 45 wt % SiO₂, 24.5 wt % Na₂O and 6 wt % P₂O₅.

### Comparative Example 5 to 6

The methods used in Comparative Example 5 to 6 are substantially the same as the method of Comparative Example 4, except that the consumption of CaCO₃, SiO₂ and Na₂CO₃ in the glass component is changed.

### [Comparative Example 7] Glass Composite and Glass Ceramic

33.48 wt % CaCO₃, 34.46 wt % SiO₂ and 32.07 wt % Na₂CO₃ are mixed to obtain a glass component, and then 100 parts by weight of the glass component and 4.59 parts by weight of P₂O₅ are mixed, and a ball milling process is performed to obtain a plurality of glass powder, and the glass powder is put into an oven, and a drying process is performed at 150° C. to obtain a glass composite.

The glass composite is put into a furnace which is set to 1500° C., and a melting and thermal decomposition is performed for 3 hours to obtain a clarified melted mixture containing CaO, SiO₂, Na₂O and P₂O₅, and then the clarified melted mixture is quickly removed from the furnace and poured into deionized water at 25° C., and a water quenching process is performed to obtain a plurality of amorphous quenched glass lumps, and then the amorphous quenched glass lumps are mixed with water and ethanol, and a zirconium ball is used to perform a crushing process to obtain a plurality of glass cullets with a particle size smaller than 50 μm, and then the glass cullets are mixed with polyvinyl alcohol, and a forming process is performed to obtain a plurality of glass ingots with a size greater than 50 μm and smaller than 1 mm, and the glass ingots are sintered at 1050° C. for 2 hours to obtain a glass ceramic containing 24.5 wt % CaO, 45 wt % SiO₂, 24.5 wt % Na₂O and 6 wt % P₂O₅.

### Comparative Example 8 to 9

The methods used in Comparative Example 8 to 9 are substantially the same as the method of Comparative Example 7, except that the consumption of CaCO₃, SiO₂ and Na₂CO₃ in the glass component is changed.

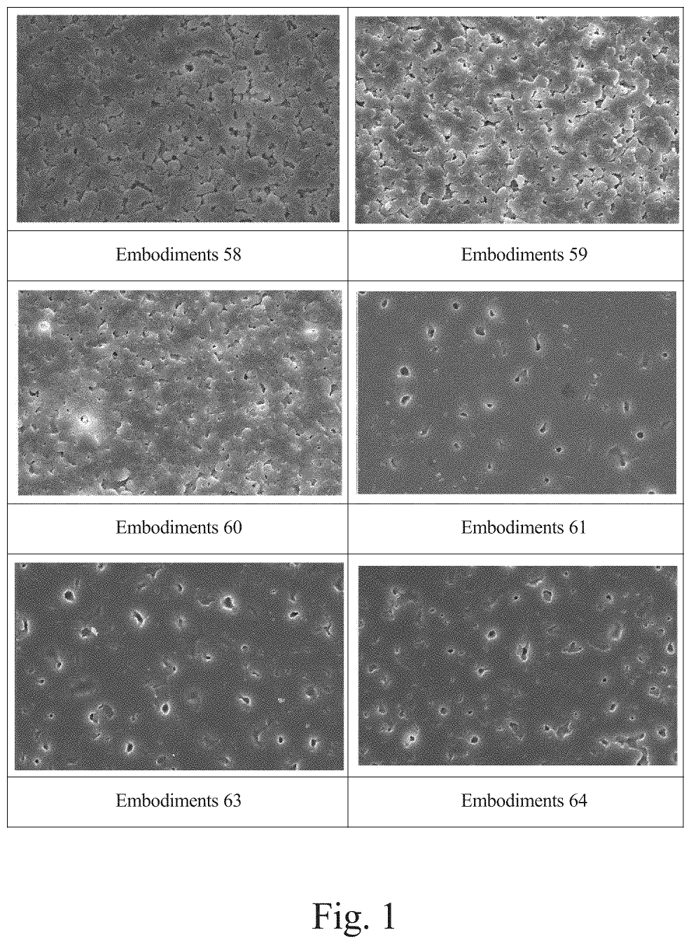

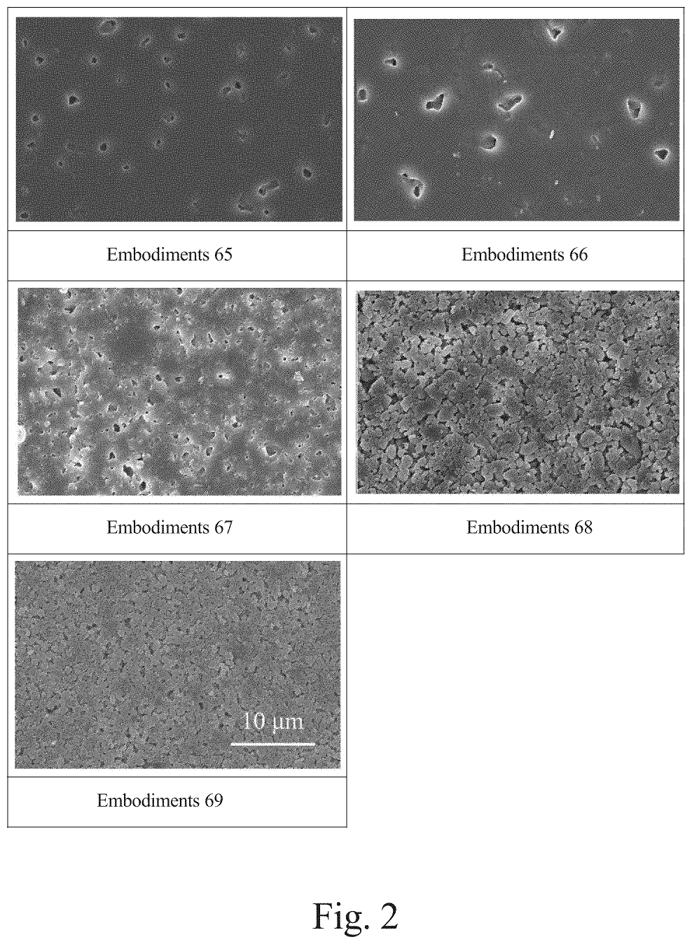

The surface density of the glass ceramic is measured by an electron microscope (Manufacturer: JEOL; Model No.: JSM-7610FPlus) to take the photos of the glass ceramics of Embodiment 58 to 61 and 63 to 69 and observe the surface conditions, and the results are shown in FIGS. 1 and 2.

In FIGS. 1 and 2, the glass ceramics of Embodiments 58 to 61 and 63 to 69 have small quantity of holes formed on the surface of the glass ceramic, and these holes have a small diameter, indicating that the surface of the glass ceramics in accordance with Embodiment 58 to 61 and 63 to 69 has a high density. Further, the number of holes formed on the surface of the glass ceramics of Embodiments 61, 63, 64, 65 and 66 is less than those of other embodiments, indicating that the surface of the glass ceramics of Embodiments 61, 63, 64, 65 and 66 has a higher density.

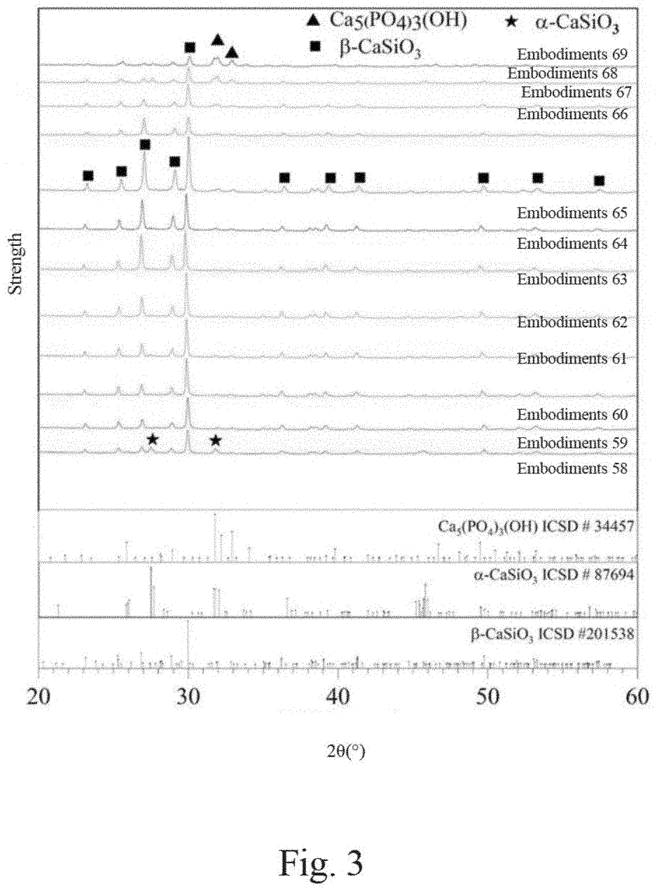

In a crystal structural analysis of the glass ceramic, an X-ray diffractometer (Manufacturer: Bruker; Model No.: D8 ADVANCE ECO) is used to measure the glass ceramics of Embodiments 58 to 69, and the results are shown in FIG. 3.

In FIG. 3, at least one characteristic peak in alpha-wollastonite (α-CaSiO₃), beta-wollastonite (β-CaSiO₃) and hydroxyapatite [Ca₅(PO₄)₃(OH)] can be observed from any one of the X-ray diffraction patterns of the glass ceramics of Embodiment 58 to 69, indicating that the glass ceramics of Embodiment 58 to 69 have at least one crystal structure in alpha-wollastonite (α-CaSiO₃), beta-wollastonite (β-CaSiO₃) and hydroxyapatite [Ca₅(PO₄)₃(OH)].

The compressive strength and flexural strength are measured by an electromechanical test system (Manufacturer: MTS Insight; Model No.: MTS Insight 5), and the glass ceramics of Embodiments 1 to 69 are measured, and the results are listed in Tables 1 to 7.

In the measurement of cell survival rate, the measuring procedure described in Embodiment 1 is used as an example, and the remaining embodiments and comparative examples are measured by this measuring procedure. Three samples of the glass ceramic of Embodiment 1 are put into a solution containing 5×10³ mouse embryonic fibroblasts L929 [Source: American Type Culture Collection (ATCC)], and cultured under a humid environment at a temperature of 37° C. and a CO₂ content of 5% for three days. In addition, the three solutions containing 5×10³ mouse embryonic fibroblasts L929 are cultured under a humid environment at a temperature of 37° C. and a CO₂ content of 5% for three days, and used as a control group, and then 3-(4,5-dimethylthiazol-2-yl)-2,5-diphenyltetrazolium bromide (MTT) is added into the solutions and reacted for 1 hour to obtain a solution of formazan, and then dimethyl sulfoxide is added into the solutions containing formazan to obtain three first solutions to be tested which contain the glass ceramic of Embodiment 1 and three second solution to be tested which contain the glass ceramic of Embodiment 1, and then a microplate reader (Manufacturer: TECAN; Model No.: Infinite 200 PRO) is used to measure the absorbance of the first solutions to be tested and the second solutions to be tested at the wavelength of 570 nm, and the average absorbance of the first solutions to be tested and the average absorbance of the second solutions to be tested are used to calculate the cell survival rate. The cell survival rates of the glass ceramics of Embodiments 1 to 69 are listed in Tables 1 to 7.

Cell survival rate (unit: %)=(Average absorbance of the first solutions to be tested/average absorbance of the second solutions to be tested)×100%.

In the measurement of osteoblast differentiation performance: the measuring procedure of Embodiment 1 is used as an example, and the remaining embodiments and comparative example are measured by this measuring procedure. Two samples of the glass ceramics of Embodiment 1 are put into first solutions containing mouse bone marrow stem cells and osteoblast differentiation media respectively, and a second solution containing mouse bone marrow stem cells and a second solution not containing osteoblast differentiation media respectively, and cultured under humid environment at a temperature of 37° C. and a CO₂ content of 5% for 4 days to obtain first culture corresponding to the first solution and a second culture corresponding to the second solution respectively, and then the mouse bone marrow stem cells in the first culture and the second culture are rinsed by phosphate buffered saline for three times, and 100 μL of paraformaldehyde solution with a concentration of 4 wt % paraformaldehyde are added, and a cell fixation process is performed for 30 minutes to obtain a first mixed solution corresponding to the first culture and a second mixed solution corresponding to the second culture, and then the mouse bone marrow stem cells fixed in the first mixed solution and the second mixed solution are rinsed by phosphate buffered saline for three times, and then 100 μL of alkaline phosphatase are added into p-nitrophenyl phosphate and the mouse bone marrow stem cells and reacted in a dark condition for 30 minutes. After the reaction ends, 25 μL of sodium hydroxide solution with a concentration of 3N are added to end the reaction and obtain a first solution to be tested corresponding to the first mixed solution and a second solution to be tested corresponding to the second mixed solution, and then a microplate reader (Manufacturer: TECAN; Model No.: Infinite 200 PRO) is used to measure the absorbance of the first solution to be tested and the absorbance of the second solution to be tested at a wavelength of 405 nm, and the absorbance of the first solution to be tested and the absorbance of the second solution to be tested are used to calculate the activity of alkaline phosphatase. The activities of alkaline phosphatase of the glass ceramics of Embodiments 1 to 69 are listed in Tables 1 to 7.

Activity of alkaline phosphatase (unit: %)=(Absorbance of first solution to be tested/Absorbance of second solution to be tested)×100%.

TABLE 1

| | | Embodiment | | | | | | | | | | |
|---|---|---|---|---|---|---|---|---|---|---|---|---|
| | | 1 | 2 | 3 | 4 | 5 | 6 | 7 | 8 | 9 | 10 | 11 |
| Glass | CaCO₃ | 62.49 | 62.49 | 62.49 | 62.49 | 62.49 | 62.49 | 62.49 | 62.49 | 62.49 | 62.49 | 62.49 |
| composite | (wt %) | | | | | | | | | | | |
| | SiO₂ | 37.51 | 37.51 | 37.51 | 37.51 | 37.51 | 37.51 | 37.51 | 37.51 | 37.51 | 37.51 | 37.51 |
| | (wt %) | | | | | | | | | | | |
| | Na₂CO₃ | 0 | 0 | 0 | 0 | 0 | 0 | 0 | 0 | 0 | 0 | 0 |
| | (wt %) | | | | | | | | | | | |
| | Total quantity | 100 | | | | | | | | | | |
| | of glass | | | | | | | | | | | |
| | ceramic | | | | | | | | | | | |
| | (parts by | | | | | | | | | | | |
| | weight) | | | | | | | | | | | |
| | P₂O₅ | 0.1 | 1 | 2 | 3 | 4 | 5 | 6 | 7 | 8 | 9 | 10 |
| | (parts by | | | | | | | | | | | |
| | weight) | | | | | | | | | | | |
| Glass | CaO | 48.21 | 47.62 | 46.98 | 46.36 | 45.75 | 45.16 | 44.59 | 44.03 | 43.48 | 42.95 | 42.43 |
| ceramic | (wt %) | | | | | | | | | | | |
| | SiO₂ | 51.65 | 51.02 | 50.34 | 49.67 | 49.02 | 48.39 | 47.77 | 47.17 | 46.58 | 46.01 | 45.46 |
| | (wt %) | | | | | | | | | | | |
| | Na₂O | 0 | 0 | 0 | 0 | 0 | 0 | 0 | 0 | 0 | 0 | 0 |
| | (wt %) | | | | | | | | | | | |
| | P₂O₅ | 0.14 | 1.36 | 2.68 | 3.97 | 5.23 | 6.45 | 7.64 | 8.80 | 9.94 | 11.04 | 12.12 |
| | (wt %) | | | | | | | | | | | |
| | Compressive | 56 | 64 | 78 | 82 | 94 | 93 | 95 | 93 | 90 | 88 | 89 |
| | strength | | | | | | | | | | | |
| | (MPa) | | | | | | | | | | | |
| | Flexural | 12 | 16 | 21 | 24 | 25 | 24 | 25 | 24 | 23 | 23 | 22 |
| | strength | | | | | | | | | | | |
| | (MPa) | | | | | | | | | | | |
| | Cell survival | 87 | 93 | 96 | 103 | 105 | 112 | 119 | 127 | 123 | 122 | 113 |
| | rate (%) | | | | | | | | | | | |
| | Activity of | 98 | 102 | 104 | 110 | 117 | 120 | 138 | 142 | 145 | 138 | 132 |
| | alkaline | | | | | | | | | | | |
| | phosphatase | | | | | | | | | | | |
| | (%) | | | | | | | | | | | |

TABLE 2

| | | | | | | | | | | Comparative | | |
|---|---|---|---|---|---|---|---|---|---|---|---|---|
| | | Embodiment | | | | | | | | Example | | |
| | | 12 | 13 | 14 | 15 | 16 | 17 | 18 | 19 | 1 | 2 | 3 |
| Glass | CaCO₃ | 62.49 | 62.49 | 62.49 | 62.49 | 62.49 | 62.49 | 62.49 | 62.49 | 33.48 | 66 | 54 |
| composite | (wt %) | | | | | | | | | | | |
| | SiO₂ | 37.51 | 37.51 | 37.51 | 37.51 | 37.51 | 37.51 | 37.51 | 37.51 | 34.46 | 34 | 46 |
| | (wt %) | | | | | | | | | | | |
| | Na₂CO₃ | 0 | 0 | 0 | 0 | 0 | 0 | 0 | 0 | 32.07 | 0 | 0 |
| | (wt %) | | | | | | | | | | | |
| | Total quantity | 100 | | | | | | | | | | |
| | of glass | | | | | | | | | | | |
| | component | | | | | | | | | | | |
| | (parts by | | | | | | | | | | | |
| | weight) | | | | | | | | | | | |
| | P₂O₅ | 11 | 12 | 13 | 14 | 15 | 16 | 17 | 18 | 4.59 | 4 | 4 |
| | (parts by | | | | | | | | | | | |
| | weight) | | | | | | | | | | | |
| Glass | CaO | 41.92 | 41.42 | 40.94 | 40.47 | 40.00 | 39.55 | 39.11 | 38.68 | 24.5 | 49.32 | 37.7 |
| ceramic | (wt %) | | | | | | | | | | | |
| | SiO₂ | 44.91 | 44.38 | 43.86 | 43.35 | 42.86 | 42.37 | 41.90 | 41.44 | 45 | 45.35 | 57.32 |
| | (wt %) | | | | | | | | | | | |
| | Na₂O | 0 | 0 | 0 | 0 | 0 | 0 | 0 | 0 | 24.5 | 0 | 0 |
| | (wt %) | | | | | | | | | | | |
| | P₂O₅ | 13.17 | 14.20 | 15.20 | 16.18 | 17.14 | 18.08 | 18.99 | 19.88 | 6 | 5.33 | 4.98 |
| | (wt %) | | | | | | | | | | | |
| | Compressive | 86 | 81 | 72 | 62 | 58 | 52 | 46 | 49 | 11 | 21 | 13 |
| | strength | | | | | | | | | | | |
| | (MPa) | | | | | | | | | | | |
| | Flexural | 22 | 21 | 20 | 19 | 18 | 15 | 14 | 13 | 8 | 7 | 8 |
| | strength | | | | | | | | | | | |
| | (MPa) | | | | | | | | | | | |
| | Cell survival | 115 | 116 | 118 | 103 | 104 | 93 | 87 | 80 | 23 | 56 | 52 |
| | rate (%) | | | | | | | | | | | |
| | Activity of | 130 | 128 | 118 | 113 | 116 | 108 | 98 | 93 | 61 | 67 | 63 |
| | alkaline | | | | | | | | | | | |
| | phosphatase | | | | | | | | | | | |
| | (%) | | | | | | | | | | | |

TABLE 3

| | | Embodiment | | | | | | | | | | |
|---|---|---|---|---|---|---|---|---|---|---|---|---|
| | | 20 | 21 | 22 | 23 | 24 | 25 | 26 | 27 | 28 | 29 | 30 |
| Glass | CaCO₃ | 62.49 | 62.49 | 62.49 | 62.49 | 62.49 | 62.49 | 62.49 | 62.49 | 62.49 | 62.49 | 62.49 |
| composite | (wt %) | | | | | | | | | | | |
| | SiO₂ | 37.51 | 37.51 | 37.51 | 37.51 | 37.51 | 37.51 | 37.51 | 37.51 | 37.51 | 37.51 | 37.51 |
| | (wt %) | | | | | | | | | | | |
| | Na₂CO₃ | 0 | 0 | 0 | 0 | 0 | 0 | 0 | 0 | 0 | 0 | 0 |
| | (wt %) | | | | | | | | | | | |
| | Total quantity | 100 | | | | | | | | | | |
| | of glass | | | | | | | | | | | |
| | component | | | | | | | | | | | |
| | (parts by | | | | | | | | | | | |
| | weight) | | | | | | | | | | | |
| | P₂O₅ | 0.1 | 1 | 2 | 3 | 4 | 5 | 6 | 7 | 8 | 9 | 10 |
| | (parts by | | | | | | | | | | | |
| | weight) | | | | | | | | | | | |
| Glass | CaO | 48.21 | 47.62 | 46.98 | 46.36 | 45.75 | 45.16 | 44.59 | 44.03 | 43.48 | 42.95 | 42.43 |
| ceramic | (wt %) | | | | | | | | | | | |
| | SiO₂ | 51.65 | 51.02 | 50.34 | 49.67 | 49.02 | 48.39 | 47.77 | 47.17 | 46.58 | 46.01 | 45.46 |
| | (wt %) | | | | | | | | | | | |
| | Na₂O | 0 | 0 | 0 | 0 | 0 | 0 | 0 | 0 | 0 | 0 | 0 |
| | (wt %) | | | | | | | | | | | |
| | P₂O₅ | 0.14 | 1.36 | 2.68 | 3.97 | 5.23 | 6.45 | 7.64 | 8.80 | 9.94 | 11.04 | 12.12 |
| | (wt %) | | | | | | | | | | | |
| | Compressive | 425 | 430 | 447 | 452 | 467 | 476 | 483 | 514 | 533 | 542 | 544 |
| | strength | | | | | | | | | | | |
| | (MPa) | | | | | | | | | | | |
| | Flexural | 102 | 114 | 117 | 121 | 132 | 136 | 137 | 135 | 136 | 135 | 133 |
| | strength | | | | | | | | | | | |
| | (MPa) | | | | | | | | | | | |
| | Cell survival | 98 | 106 | 118 | 123 | 132 | 142 | 152 | 162 | 142 | 133 | 131 |
| | rate (%) | | | | | | | | | | | |
| | Activity of | 138 | 147 | 165 | 179 | 202 | 237 | 269 | 273 | 248 | 231 | 204 |
| | alkaline | | | | | | | | | | | |
| | phosphatase | | | | | | | | | | | |
| | (%) | | | | | | | | | | | |

TABLE 4

| | | Embodiment | | | | | | | | Comparative Example | | |
|---|---|---|---|---|---|---|---|---|---|---|---|---|
| | | 31 | 32 | 33 | 34 | 35 | 36 | 37 | 38 | 4 | 5 | 6 |
| Glass | CaCO₃ | 62.49 | 62.49 | 62.49 | 62.49 | 62.49 | 62.49 | 62.49 | 62.49 | 33.48 | 66 | 54 |
| composite | (wt %) | | | | | | | | | | | |
| | SiO₂ | 37.51 | 37.51 | 37.51 | 37.51 | 37.51 | 37.51 | 37.51 | 37.51 | 34.46 | 34 | 46 |
| | (wt %) | | | | | | | | | | | |
| | Na₂CO₃ | 0 | 0 | 0 | 0 | 0 | 0 | 0 | 0 | 32.07 | 0 | 0 |
| | (wt %) | | | | | | | | | | | |
| | Total quantity | 100 | | | | | | | | | | |
| | of glass | | | | | | | | | | | |
| | component | | | | | | | | | | | |
| | (parts by | | | | | | | | | | | |
| | weight) | | | | | | | | | | | |
| | P₂O₅ | 11 | 12 | 13 | 14 | 15 | 16 | 17 | 18 | 4.59 | 4 | 4 |
| | (parts by | | | | | | | | | | | |
| | weight) | | | | | | | | | | | |
| Glass | CaO | 41.92 | 41.42 | 40.94 | 40.47 | 40.00 | 39.55 | 39.11 | 38.68 | 24.5 | 49.32 | 37.7 |
| ceramic | (wt %) | | | | | | | | | | | |
| | SiO₂ | 44.91 | 44.38 | 43.86 | 43.35 | 42.86 | 42.37 | 41.90 | 41.44 | 45 | 45.35 | 57.32 |
| | (wt %) | | | | | | | | | | | |
| | Na₂O | 0 | 0 | 0 | 0 | 0 | 0 | 0 | 0 | 24.5 | 0 | 0 |
| | (wt %) | | | | | | | | | | | |
| | P₂O₅ | 13.17 | 14.20 | 15.20 | 16.18 | 17.14 | 18.08 | 18.99 | 19.88 | 6 | 5.33 | 4.98 |
| | (wt %) | | | | | | | | | | | |
| | Compressive | 532 | 506 | 483 | 476 | 462 | 451 | 430 | 413 | 370 | 310 | 330 |
| | strength | | | | | | | | | | | |
| | (MPa) | | | | | | | | | | | |
| | Flexural | 134 | 132 | 125 | 118 | 110 | 104 | 103 | 100 | 27 | 32 | 35 |
| | strength | | | | | | | | | | | |
| | (MPa) | | | | | | | | | | | |
| | Cell survival | 121 | 118 | 101 | 108 | 110 | 105 | 97 | 93 | 45 | 65 | 62 |
| | rate (%) | | | | | | | | | | | |
| | Activity of | 193 | 183 | 178 | 142 | 131 | 126 | 119 | 102 | 80 | 83 | 82 |
| | alkaline | | | | | | | | | | | |
| | phosphatase | | | | | | | | | | | |
| | (%) | | | | | | | | | | | |

TABLE 5

| | | Embodiment | | | | | | | | | | |
|---|---|---|---|---|---|---|---|---|---|---|---|---|
| | | 39 | 40 | 41 | 42 | 43 | 44 | 45 | 46 | 47 | 48 | 49 |
| Glass | CaCO₃ | 62.49 | 62.49 | 62.49 | 62.49 | 62.49 | 62.49 | 62.49 | 62.49 | 62.49 | 62.49 | 62.49 |
| composite | (wt %) | | | | | | | | | | | |
| | SiO₂ | 37.51 | 37.1 | 37.51 | 37.51 | 37.51 | 37.51 | 37.51 | 37.51 | 37.51 | 37.51 | 37.51 |
| | (wt %) | | | | | | | | | | | |
| | Na₂CO₃ | 0 | 0 | 0 | 0 | 0 | 0 | 0 | 0 | 0 | 0 | 0 |
| | (wt %) | | | | | | | | | | | |
| | Total quantity | 100 | | | | | | | | | | |
| | of glass | | | | | | | | | | | |
| | component | | | | | | | | | | | |
| | (parts by | | | | | | | | | | | |
| | weight) | | | | | | | | | | | |
| | P₂O₅ | 0.1 | 1 | 2 | 3 | 4 | 5 | 6 | 7 | 8 | 9 | 10 |
| | (parts by | | | | | | | | | | | |
| | weight) | | | | | | | | | | | |
| Glass | CaO | 48.21 | 47.62 | 46.98 | 46.36 | 45.75 | 45.16 | 44.59 | 44.03 | 43.48 | 42.95 | 42.43 |
| ceramic | (wt %) | | | | | | | | | | | |
| | SiO₂ | 51.65 | 51.02 | 50.34 | 49.67 | 49.02 | 48.39 | 47.77 | 47.17 | 46.58 | 46.01 | 45.46 |
| | (wt %) | | | | | | | | | | | |
| | Na₂O | 0 | 0 | 0 | 0 | 0 | 0 | 0 | 0 | 0 | 0 | 0 |
| | (wt %) | | | | | | | | | | | |
| | P₂O₅ | 0.14 | 1.36 | 2.68 | 3.97 | 5.23 | 6.45 | 7.64 | 8.80 | 9.94 | 11.04 | 12.12 |
| | (wt %) | | | | | | | | | | | |
| | Compressive | 876 | 954 | 989 | 1008 | 1110 | 1123 | 1178 | 1169 | 1138 | 1128 | 1110 |
| | strength | | | | | | | | | | | |
| | (MPa) | | | | | | | | | | | |
| | Flexural | 133 | 138 | 142 | 150 | 156 | 160 | 167 | 160 | 152 | 150 | 149 |
| | strength | | | | | | | | | | | |
| | (MPa) | | | | | | | | | | | |
| | Cell survival | 103 | 119 | 129 | 132 | 137 | 143 | 157 | 167 | 153 | 143 | 142 |
| | rate (%) | | | | | | | | | | | |
| | Activity of | 152 | 162 | 173 | 189 | 220 | 273 | 312 | 311 | 284 | 273 | 243 |
| | alkaline | | | | | | | | | | | |
| | phosphatase | | | | | | | | | | | |
| | (%) | | | | | | | | | | | |

TABLE 6

| | | | | | | | | | | Comparative | | |
|---|---|---|---|---|---|---|---|---|---|---|---|---|
| | | Embodiment | | | | | | | | Example | | |
| | | 50 | 51 | 52 | 53 | 54 | 55 | 56 | 57 | 7 | 8 | 9 |
| glass | CaCO₃ | 62.49 | 62.49 | 62.49 | 62.49 | 62.49 | 62.49 | 62.49 | 62.49 | 33.48 | 66 | 54 |
| composite | (wt %) | | | | | | | | | | | |
| | SiO₂ | 37.51 | 37.51 | 37.51 | 37.51 | 37.51 | 37.51 | 37.51 | 37.51 | 34.46 | 34 | 46 |
| | (wt %) | | | | | | | | | | | |
| | Na₂CO₃ | 0 | 0 | 0 | 0 | 0 | 0 | 0 | 0 | 32.07 | 0 | 0 |
| | (wt %) | | | | | | | | | | | |
| | Total quantity | 100 | | | | | | | | | | |
| | of glass | | | | | | | | | | | |
| | component | | | | | | | | | | | |
| | (parts by | | | | | | | | | | | |
| | weight) | | | | | | | | | | | |
| | P₂O₅ | 11 | 12 | 13 | 14 | 15 | 16 | 17 | 18 | 4.59 | 4 | 4 |
| | (parts by | | | | | | | | | | | |
| | weight) | | | | | | | | | | | |
| glass | CaO | 41.92 | 41.42 | 40.94 | 40.47 | 40.00 | 39.55 | 39.11 | 38.68 | 24.5 | 49.32 | 37.7 |
| ceramic | (wt %) | | | | | | | | | | | |
| | SiO₂ | 44.91 | 44.38 | 43.86 | 43.35 | 42.86 | 42.37 | 41.90 | 41.44 | 45 | 45.35 | 57.32 |
| | (wt %) | | | | | | | | | | | |
| | Na₂O | 0 | 0 | 0 | 0 | 0 | 0 | 0 | 0 | 24.5 | 0 | 0 |
| | (wt %) | | | | | | | | | | | |
| | P₂O₅ | 13.17 | 14.20 | 15.20 | 16.18 | 17.14 | 18.08 | 18.99 | 19.88 | 6 | 5.33 | 4.98 |
| | (wt %) | | | | | | | | | | | |
| | Compressive | 1103 | 1005 | 993 | 987 | 976 | 965 | 955 | 932 | 531 | 542 | 523 |
| | strength | | | | | | | | | | | |
| | (MPa) | | | | | | | | | | | |
| | Flexural | 146 | 142 | 140 | 138 | 135 | 130 | 129 | 125 | 43 | 62 | 56 |
| | strength | | | | | | | | | | | |
| | (MPa) | | | | | | | | | | | |
| | Cell survival | 136 | 128 | 121 | 117 | 108 | 103 | 100 | 98 | 50 | 75 | 72 |
| | rate (%) | | | | | | | | | | | |
| | Activity of | 204 | 187 | 174 | 158 | 136 | 124 | 122 | 108 | 90 | 93 | 92 |
| | alkaline | | | | | | | | | | | |
| | phosphatase | | | | | | | | | | | |
| | (%) | | | | | | | | | | | |

TABLE 7

| | | Embodiment | | | | | | | | | | | |
|---|---|---|---|---|---|---|---|---|---|---|---|---|---|
| | | 58 | 59 | 60 | 61 | 62 | 63 | 64 | 65 | 66 | 67 | 68 | 69 |
| glass | CaCO₃ | 62.49 | 62.49 | 62.49 | 62.49 | 62.49 | 62.49 | 62.49 | 62.49 | 62.49 | 62.49 | 62.49 | 62.49 |
| composite | (wt %) | | | | | | | | | | | | |
| | SiO₂ | 37.51 | 37.51 | 37.51 | 37.51 | 37.51 | 37.51 | 37.51 | 37.51 | 37.51 | 37.51 | 37.51 | 37.51 |
| | (wt %) | | | | | | | | | | | | |
| | Na₂CO₃ | 0 | 0 | 0 | 0 | 0 | 0 | 0 | 0 | 0 | 0 | 0 | 0 |
| | (wt %) | | | | | | | | | | | | |
| | Total quantity | 100 | | | | | | | | | | | |
| | of glass | | | | | | | | | | | | |
| | component | | | | | | | | | | | | |
| | (parts by | | | | | | | | | | | | |
| | weight) | | | | | | | | | | | | |
| | P₂O₅ | 0.5 | 1 | 2.5 | 3 | 3.5 | 4 | 4.5 | 5 | 5.5 | 6 | 7.5 | 10 |
| | (parts by | | | | | | | | | | | | |
| | weight) | | | | | | | | | | | | |
| glass | CaO | 47.95 | 47.62 | 46.67 | 46.36 | 46.06 | 45.75 | 45.46 | 45.16 | 44.87 | 44.59 | 43.75 | 42.43 |
| ceramic | (wt %) | | | | | | | | | | | | |
| | SiO₂ | 51.37 | 51.02 | 50.00 | 49.67 | 49.34 | 49.02 | 48.70 | 48.39 | 48.08 | 47.77 | 46.88 | 45.46 |
| | (wt %) | | | | | | | | | | | | |
| | Na₂O | 0 | 0 | 0 | 0 | 0 | 0 | 0 | 0 | 0 | 0 | 0 | 0 |
| | (wt %) | | | | | | | | | | | | |
| | P₂O₅ | 0.68 | 1.36 | 3.33 | 3.97 | 4.60 | 5.23 | 5.84 | 6.45 | 7.05 | 7.64 | 9.37 | 12.12 |
| | (wt %) | | | | | | | | | | | | |
| | Compressive | 765 | 892 | 920 | 989 | 1029 | 1166 | 1178 | 1158 | 1031 | 920 | 831 | 787 |
| | strength | | | | | | | | | | | | |
| | (MPa) | | | | | | | | | | | | |
| | Flexural | 103 | 128 | 134 | 148 | 152 | 166 | 167 | 163 | 152 | 148 | 131 | 117 |
| | strength | | | | | | | | | | | | |
| | (MPa) | | | | | | | | | | | | |
| | Cell survival | 104 | 119 | 128 | 133 | 142 | 150 | 142 | 132 | 123 | 110 | 110 | 100 |
| | rate (%) | | | | | | | | | | | | |
| | Activity of | 152 | 171 | 182 | 234 | 274 | 312 | 283 | 277 | 231 | 204 | 178 | 163 |
| | alkaline | | | | | | | | | | | | |
| | phosphatase | | | | | | | | | | | | |
| | (%) | | | | | | | | | | | | |

In Tables 1 to 7, with the combination of CaO, SiO₂ and P₂O₅, and the control of CaO content within a range from 38 wt % to 49 wt %, the control of SiO₂ content within a range from 41 wt % to 52 wt % and the control of P₂O₅ content within a range from 0.1 wt % to 20 wt %, the glass ceramics of Embodiment 1 to 69 have high compressive strength and high flexural strength, indicating that the glass ceramics of Embodiments 1 to 69 will not be deformed, broken or cracked easily by the impacts of external forces, so that the glass ceramics of Embodiments 1 to 69 are suitable to be used as a fixation bracket for defect tissue repair. In addition, the glass composite of this disclosure does not contain alkali metals, and the cell survival rates listed in Tables 1 to 7 show that after the glass ceramics of Embodiments 1 to 69 are react with the mouse embryonic fibroblasts L929, the mouse embryonic fibroblasts L929 still have a cell survival rate greater than 80%, indicating that the glass ceramics have low cytotoxicity and high biocompatibility.

On the other hand, the glass ceramics of Comparative Examples 1, 4 and 7 among the nine comparative examples do not control the CaO content within the range from 38 wt % to 49 wt %, which is equivalent to Bioglass®45S5, so that the glass ceramics of Comparative Examples 1, 4 and 7 have low compressive strength and low flexural strength. In addition, the glass ceramics of Comparative Example 1, 4 and 7 contain alkali metal oxides, so that after the glass ceramics of Comparative Example 1, 4 and 7 are reacted with the mouse embryonic fibroblasts L929, the mouse embryonic fibroblasts L929 only have a cell survival rate below 50%, indicating that the glass ceramics of Comparative Example 1, 4 and 7 have high cytotoxicity and low biocompatibility.

The glass ceramics of Comparative Examples 2 to 3, Comparative Examples 5 to 6 and Comparative Examples 8 to 9 do not control the CaO content within a range from 38 wt % to 49 wt %, so that the glass ceramics of Comparative Examples 2 to 3, Comparative Examples 5 to 6 and Comparative Examples 8 to 9 have low compressive strength and low flexural strength.

With reference to Table 1 to Table 7, the activity of alkaline phosphatase in the glass ceramics of Embodiments 1 to 69 can improve the performance of alkaline phosphatase in the mouse bone marrow stem cells, and thus these glass ceramics have a high activity of alkaline phosphatase, indicating that the glass ceramics of Embodiments 1 to 69 are conducive to osteoblast differentiation and bone growth, so that the glass ceramic of this disclosure glass ceramic can be used as a bone filling material.

In summation of the description above, the glass ceramic of this disclosure has the advantages of good compressive strength, and good flexural strength through the combination of CaO, SiO₂ and P₂O₅ and the control of CaO within a range of 38 wt % to 49 wt %, the control of SiO₂ content within a range of 41 wt % to 52 wt % and the control of P₂O₅ content within a range of 0.1 wt % to 20 wt %. In addition, the glass composite of this disclosure does not contain alkali metal oxides, so that the glass ceramic formed by the glass composite has low cytotoxicity and good biocompatibility, and surely can achieve the objectives of this disclosure.

While the disclosure has been described by means of specific embodiments, numerous modifications and variations could be made thereto by those skilled in the art without departing from the scope and spirit of the disclosure as set forth in the claims.

What is claimed is:

1. A glass ceramic, manufactured by a method comprising the steps of sequentially performing a melting and thermal decomposition, a water quenching process and a sintering process of a glass composite, and the glass ceramic comprises 38 wt % to 49 wt % CaO, 41 wt % to 52 wt % SiO₂ and 0.1 wt % to 20 wt % P₂O₅ when the total quantity of glass ceramic is considered as 100 wt %;
wherein, the glass composite comprises a glass component and P₂O₅, and the glass component comprises CaCO₃ and SiO₂ and do not comprises an alkali metal oxide, and the melting and thermal decomposition temperature falls within a range from 1350° C. to 1650° C., and the sintering temperature falls within a range from 750° C. to 1050° C.

2. The glass ceramic according to claim 1, wherein when the sintering temperature is 750° C., and the content of P₂O₅ falls within a range from 3.9 wt % to 14.2 wt % when the total quantity of glass ceramic is considered as 100 wt %.

3. The glass ceramic according to claim 1, wherein when the sintering temperature is 950° C., and the total quantity of glass ceramic is considered as 100 wt %, the content of P₂O₅ falls within a range from 3.9 wt % to 18.1 wt %.

4. The glass ceramic according to claim 1, wherein when the sintering temperature is 1050° C., and the total quantity of glass ceramic is considered as 100 wt %, the content of P₂O₅ falls within a range from 3.9 wt % to 14.2 wt %.

5. The glass ceramic according to claim 1, wherein the method further comprises a forming process performed after the water quenching process and before the sintering process, and the forming process is performed by using an adhesive.

6. The glass ceramic according to claim 5, wherein the adhesive is one selected from the group consisting of Polyvinyl alcohol, polyvinyl butyral, carboxyethyl cellulose and sodium carboxymethyl cellulose.

7. A glass composite, comprising:
a glass component, comprising CaCO₃ and SiO₂, and not comprising an alkali metal oxide; and P₂O₅;
wherein, the content of CaCO₃ falls within a range from 55 wt % to 65 wt % when the total quantity of glass component is considered as 100 wt %, and the content of P₂O₅ falls within a range from 0.1 part by weight to 18 parts by weight when the total quantity of glass component is considered as 100 parts by weight.

8. The glass composite according to claim 7, wherein the content of P₂O₅ falls within a range from 0.5 part by weight to 10 parts by weight, when the total quantity of glass component is considered as 100 parts by weight.
